(19) United States
(12) Patent Application Publication
(10) Pub. No.: US 2023/0122152 A1
(43) Pub. Date: Apr. 20, 2023

(54) ENDOLUMINAL VALVE PLACEMENT PATIENT OUTCOME PREDICTION

(71) Applicants: Matthew E. Nickeson, Redmond, WA (US); Torsten M. Lyon, St. Louis, MO (US)

(72) Inventors: Matthew E. Nickeson, Redmond, WA (US); Torsten M. Lyon, St. Louis, MO (US)

(21) Appl. No.: 18/047,736

(22) Filed: Oct. 19, 2022

Related U.S. Application Data
(60) Provisional application No. 63/262,776, filed on Oct. 20, 2021.

(51) Int. Cl. G16H 50/20; A61B 5/087; G16H 20/40

(52) U.S. Cl. CPC G16H 50/20; A61B 5/087; G16H 20/40

(57) ABSTRACT

Various aspects of methods, systems, and use cases may be used to train a model to determine whether a patient is a candidate for receiving an endoluminal valve based on collateral ventilation data. A method may include receiving sensor data based on pressure or airflow at a target portion of a lung of a patient that is occluded from receiving air via a breathing airway of the lung. The method may include training a machine learning model, based at least in part on training data (e.g., based on the sensor data), to predict patient breathing outcomes via an indication of whether collateral ventilation is present in a particular patient target lung portion.

### CLAIM OF PRIORITY

This application claims the benefit of priority to U.S. Provisional Application No. 63/262,776, filed Oct. 20, 2021, titled “ENDOLUMINAL VALVE PLACEMENT PATIENT OUTCOME PREDICTION,” which is hereby incorporated herein by reference in its entirety.

### BACKGROUND

Endoluminal valves may be placed inside airways leading to a diseased portion of a lung to redirect breathed air away from diseased areas toward healthier portions of the lung. These endoluminal valves are check valves that allow air and bodily fluids (e.g., mucus) to escape the diseased portions of the lung while preventing breathed air from entering these portions. As diseased portions of a lung (e.g., lung areas with significant emphysema) tend to increase in volume and prevent other portions from adequately expanding, endoluminal valve placement is an effective treatment for reducing the volume occupied by diseased lung portions (which do not contribute toward O₂—CO₂ gas exchange). Reducing the volume of diseased portions provides healthy lung portions with more space to fully inflate during the respiratory cycle, which allows for markedly greater gas exchange. Unfortunately, some diseased lung portions may receive airflow from collateral ventilation in which gas passes from one lung unit into a contiguous lung unit through collateral channels such as, for example, alveolar pores and/or direct airway anastomoses. Although endoluminal valve placement may be an effective treatment even with some degree of collateral ventilation present, a relatively higher degree of collateral ventilation may render endoluminal valve placement an ineffective treatment for lung volume reduction.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example Collateral Ventilation Quantification System (CVQS) in accordance with at least one example of this disclosure.

FIG. 2 illustrates a machine learning model training diagram in accordance with at least one example of this disclosure.

FIG. 3 illustrates a machine learning model inference diagram in accordance with at least one example of this disclosure.

FIG. 4A illustrates a flow and pressure diagram illustrating collateral ventilation in accordance with at least one example of this disclosure.

FIG. 4B illustrates a flow and volume diagram illustrating collateral ventilation in accordance with at least one example of this disclosure.

FIG. 4C illustrates a flow and pressure diagram illustrating a lack of collateral ventilation (no CV present) in accordance with at least one example of this disclosure.

FIG. 4D illustrates a flow and volume diagram illustrating a lack of collateral ventilation (no CV present) in accordance with at least one example of this disclosure.

FIG. 5 illustrates a flowchart showing a technique for training a model to determine whether a patient is a candidate for receiving an endoluminal valve based on collateral ventilation data in accordance with at least one example of this disclosure.

FIG. 6 illustrates a block diagram of an example machine upon which any one or more of the techniques discussed herein may perform in accordance with at least one example of this disclosure.

### DETAILED DESCRIPTION

As discussed above, some diseased lung portions may receive airflow from collateral ventilation and/or collateral channels. Depending on the degree thereof, this collateral ventilation (CV) may make an endoluminal valve placement an ineffective treatment for lung volume reduction. This is because although the endoluminal valve may function properly and prevent air from entering a diseased lung portion via a normal airway (e.g., bronchi, etc.), the collateral ventilation may be present to such a degree that air freely enters the diseased portion without passing through the normal airway in which the valve is placed. Accordingly, prior to treating a Chronic Obstructive Pulmonary Disease (COPD) patient with placement of endoluminal valves, the targeted lung regions are typically evaluated to make sure that they are not receiving airflow via collateral ventilation to such a degree that the COPD patient is unlikely to respond well to endoluminal valve placement

To evaluate the target lung regions for CV, a Collateral Ventilation Quantification System (CVQS) may be used, The CVQS may output, such as on a graphical user interface (GUI), sensor data (e.g., pressure and/or air flow). As a specific but non-limiting example, measurements of pressure within an occluded lung lobe and air flow into (or out of) the occluded lung lobe may be periodically or continuously sampled. The output may include a graph and/or data points over time. A clinician (e.g., a doctor, surgeon, specialist, etc.) may efvaluate the sensor data to determine whether CV exists for the target lung region and/or the degree of CV. However, in some examples, the clinician may not be able to determine whether CV exists, may require a long span of time to evaluate whether CV exists, and/or may not be able to precisely determine a degree of CV that exists with a high level of confidence. In some examples, even when CV exists to some degree, a patient may still benefit from placement of an endoluminal valve depending on a litany of factors such as patient age, weight, body mass index (BMI), medical history, to name a few. However, the likelihood of a positive outcome from endoluminal valve placement may not be determinable by clinician evaluation alone.

The systems and techniques described herein provide a model (e.g., a classifier, a trained model, such as using machine learning techniques, also known as artificial intelligence, or the like) to provide infoi mation related to whether a patient has CV. The model may output an indication of CV being present (CV+) or CV not being present (CV−) in a patient target lung region. In some examples, the model may output a probability and/or confidence level of whether CV exists, a degree of CV (e.g., an estimation of CV flow such as low, medium, high, a flow over time result, etc.), a degree of collateral resistance between an occluded lung portion and contiguous lung portions, or the like.

In an example, the model may output an indication of whether a patient is a good candidate for an endoluminal valve (instead of or in addition to whether CV exists). In this example, the indication may include a determination of CV, but may not be entirely dependent on whether CV exists. For example, a patient may benefit in some examples from an endoluminal valve despite having some CV.

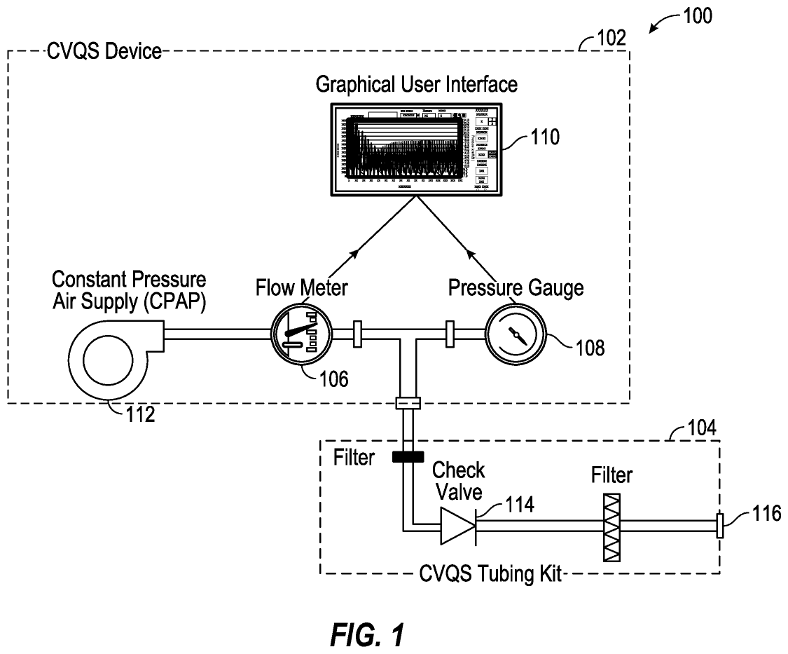

FIG. 1 illustrates an example Collateral Ventilation Quantification System (CVQS) 100 in accordance with at least one example of this disclosure. The CVQS 100 is illustrated as a positive pressure system, but in other examples, may not include a positive pressure. For example, the illustrated CVQS 100 includes air flow into a lung portion, but other examples may include a system allowing air flow out of a lung portion while preventing air flow into the lung portion. Generally speaking, in some examples, air flow in or out of a lung portion is restricted, to determine whether collateral ventilation exists in the lung portion.

The CVQS 100 includes a CVQS device 102 and a CVQS tubing kit 104. The CVQS device 102 includes a flow meter 106, a pressure gauge 108, a display device 110 (e.g., including a graphical user interface), and a constant pressure air supply 112 (e.g., a continuous positive airway pressure CPAP). In some examples, the CVQS device 102 may not include one or more of these components. For example, the constant pressure air supply 112 may not be used (and optionally the flow meter 106 and pressure gauge 108 may be omitted in this example). In an example, the display device 110 may be located remotely from the CVQS device 102 (e.g., communicatively coupled via wired or wireless communications architecture, such as ethernet, Wi-Fi, Bluetooth, etc.).

The CVQS tubing kit 104 may include various tubes and/or filters, such as a tube that is used to add air to a lung portion, and/or a tube that removes air from the lung portion. The CVQS tubing kit 104 includes a check valve 114, which may restrict air into or out of the lung portion. In the example shown in CVQS 100, the check valve 114 prevents air flow out of the lung portion, but allows air to flow into the lung portion (e.g., via the constant pressure air supply 112). In other examples, a check valve may prevent air flow into the lung portion while allowing air to exit the lung portion.

The CVQS 100 illustrated in FIG. 1 supplies positive pressure to an occluded portion of a lung and measures pressure-over-time (using the pressure gauge 108) and/or flow-over-time (using the flow meter 106). The pressure and/or flow over time may be used to assess whether the occluded lung portion is ventilating air via a collateral channel. The targeted lung portion may be occluded with an occlusion balloon (e.g., balloon catheter B7-2C by Olympus), an endoluminal valve, and/or other suitable occlusion device 116. In an example, the balloon catheter temporarily isolates a segment or segments of the lung by being inflated within the airway. While inflated in the airway, the CVQS device 102 may provide airflow through an inner lumen of the catheter, such as at a constant pressure (e.g., at 10 cm H2O). The airflow through the catheter lumen may be monitored by the flow meter 106 and/or the pressure gauge 108 of the CVQS device 102, where the data (e.g., flow, pressure, and/or total volume) may be output. The output may be displayed on the display device 110 (e.g., a portable computer, a mobile device, or the like). In some examples, the output is stored without being displayed. The output may be used for predicting a patient state (e.g., having or not having collateral ventilation, and/or being a good candidate or a poor candidate for an endoluminal valve), such as via a machine learning trained model.

The tubing kit 104 may connect the balloon catheter to the CVQS device 102. The tubing kit 104 may include a filter and check valve to allow only airflow into the target lobe (e.g., supplied by the constant pressure air supply 112) for example for one to ten minutes (e.g., three to five minutes), five to twenty minutes (e.g., approximately ten minutes), or the like. The flow through the balloon catheter may be affected by an amount of collateral ventilation within the tissue. For example, the air may continue to flow into the occluded lobe until the end-expiratory pressure equalizes with the CVQS source pressure. When collateral ventilation is present, air continues to flow into the lobe as it escapes through the collateral channels (e.g., the end pressure may not be reached or may be reached but flow may continue). When air is able to escape the occluded lobe into an adjacent lobe via a collateral ventilation channel, the lobar pressure may not equalize and/or air may continue to flow into the occluded lobe, such as via the occlusion catheter lumen and/or out of the occluded lobe via the collateral ventilation channel.

In some emphysema or other lung patients, alveoli of the lungs may become swollen and stop doing gas exchange. As the alveoli become larger, they push on another lobe causing less air to be exchanged. An endoluminal valve may be used to allow air to exit a lung portion but not flow in, thereby resulting in a reduced lung volume for the diseased portion of the lung. This type of valve may alleviate the issues associated with some symptoms of these patients. However, when collateral ventilation is present, the air that leaks between lobes via collateral channels may cause the valve to not work or be less effective. Collateral ventilation may occur between lung lobes and/or between sections of lung lobes where air travels between the lobes and/or sections. Collateral ventilation may occur in patients with emphysema. In some patients, lung fissure breaks down and causes a breakthrough between the lobes resulting in collateral ventilation. In some examples, a fissure completeness score may be used to evaluate whether a patient is a good candidate for an endoluminal valve. For example, when a fissure completeness score for a patient is above 90% or so, then the lobe may be assessed as being sufficiently intact such that a valve may be placed in the patient. In scores below 90%, more uncertainty may be present as to whether an endoluminal valve would be effective. The CVQS 100 may be used with a machine learning trained model to evaluate patients to determine whether the patient would benefit from an endoluminal valve.

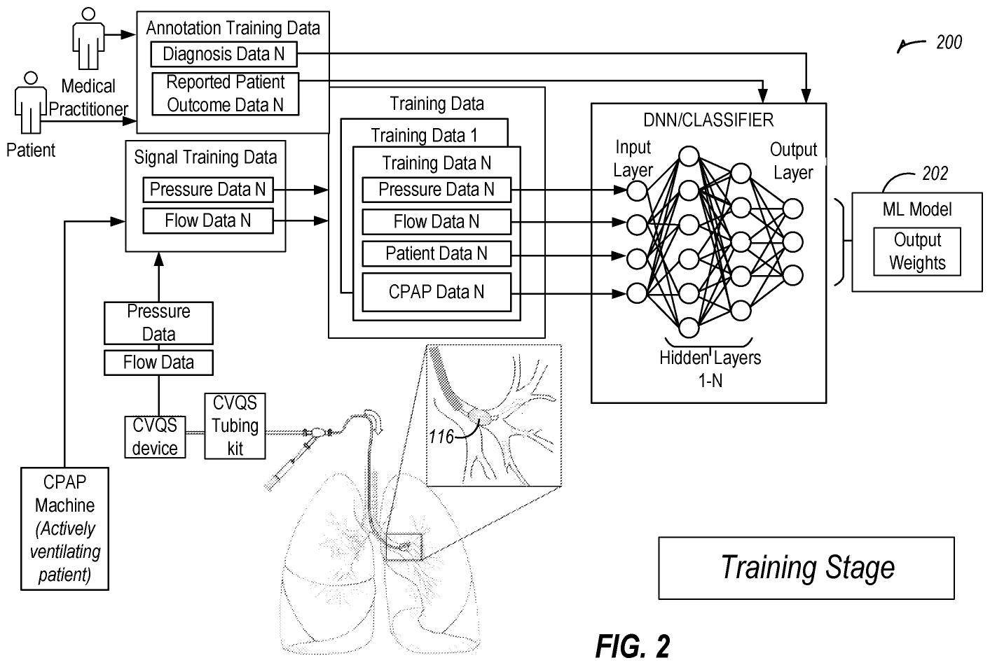

FIG. 2 illustrates a machine learning model training diagram 200 in accordance with at least one example of this disclosure. The diagram 200 illustrates components and inputs for training a model 202 using machine learning.

Machine Learning (ML) is an application that provides computer systems the ability to perform tasks, without explicitly being programmed, by making inferences based on patterns found in the analysis of data. Machine learning explores the study and construction of algorithms, also referred to herein as tools, that may learn from existing data and make predictions about new data. Although examples may be presented with respect to a few machine-learning tools, the principles presented herein may be applied to other machine-learning tools.

The machine-learning algorithms use data (e.g., action primitives and/or interaction primitives, goal vector, reward, etc.) to find correlations among identified features that affect the outcome. A feature is an individual measurable property of a phenomenon being observed. Example features for the model 202 may include diagnosis data (e.g., from a physician), reported patient outcome data, and/or other labels for patient state and/or status, with or without an endoluminal valve. The features, which may include and/or be called label data, may be compared to input data, such as pressure data, flow data, etc.

The concept of a feature is related to that of an explanatory variable used in statistical techniques such as linear regression. Choosing informative, discriminating, and independent features is important for effective operation of ML in pattern recognition, classification, and regression. Features may be of different types, such as numeric features, strings, and graphs.

During training, a ML algorithm analyzes the input data based on identified features and optionally configuration parameters defined for the training (e.g., environmental data, state data, patient data such as demographics and/or comorbidities, etc.) The result of the training is the model 202, which is capable of taking inputs to produce a complex task.

In an example, input data may be labeled (e.g., for use as features in a training stage). Labeling may include identifying patient state and/or status after a procedure and/or after no procedure. For example, patient state and/or status may be labeled as including an endoluminal valve intervention or not. The patient state and/or status may include an objective outcome (e.g., CV was present or not, patient breathing improved based on an objective test, etc.) and/or subjective outcome (e.g., patient perceives an improvement to breathing and/or quality of life, clinician assesses whether CV was present or not such as using a visual determination, etc.). The outcomes may be identified over time, such as in three and/or six months after an intervention (or no intervention). The time labels may be weighted, and/or may be used to generate different versions of the model 202. An example objective outcome includes a fissure integrity score. The score may be weighted, such as at 90 or better being more indicative of no CV and 80 or lower being indicative of CV. In some examples, a label may include a weighting for a degree of CV. In these examples, the weighting may be based on whether there is a little flow or a lot of flow to improve the model 202. Some outcomes discussed herein may be used to update the model 202 after initial training.

Input training data for the model 202 may include pressure data and/or flow data as discussed above. Other data used for input data may include a CT scan (e.g., high resolution), such as with a corresponding a Fissure Integrity Score, a disease state, patient age, confounding factors such as infection, or the like.

A neural network, sometimes referred to as an artificial neural network, is a computing system based on consideration of biological neural networks of animal brains. Such systems progressively improve performance, which is referred to as learning, to perform tasks, typically without task-specific programming, For example, in image recognition, a neural network may be taught to identify images that contain an object by analyzing example images that have been tagged with a name for the object, and having learned the object and name, may use the analytic results to identify and/or classify the object in untagged images. In FIG. 2 for example, the model 202 may be trained to identify whether a patient has CV or not based on input data (e.g., pressure and/or flow) and/or classify a patient (e.g., as a candidate for an endoluminal valve or not, and/or with a percentage likelihood and/or confidence of success of an endoluminal valve procedure).

A neural network is based on a collection of connected units called neurons, where each connection, called a synapse, between neurons can transmit a unidirectional signal with an activating strength that varies with the strength of the connection. The receiving neuron can activate and propagate a signal to downstream neurons connected to it, typically based on whether the combined incoming signals, which are from potentially many transmitting neurons, are of sufficient strength, where strength is a parameter.

A deep neural network (DNN) is a stacked neural network, which is composed of multiple layers. The layers are composed of nodes, which are locations where computation occurs, loosely patterned on a neuron in the human brain, which fires when it encounters sufficient stimuli. A node combines input from the data with a set of coefficients, and/or weights, that either amplify or dampen that input, which assigns significance to inputs for the task the algorithm is trying to learn. These input-weight products are summed, and the sum is passed through what is called an activation function for a node, to determine whether and to what extent that signal progresses further through the network to affect the ultimate outcome. A DNN uses a cascade of many layers of non-linear processing units for feature extraction and transformation. Each successive layer uses the output from the previous layer as input. Higher-level features are derived from lower-level features to form a hierarchical representation. The layers following the input layer may be convolution layers that produce feature maps that are filtering results of the inputs and are used by the next convolution layer.

The DNN may be a specific type of DNN, such as a convolutional neural network (CNN), a recurrent neural network (RNN), a Long Short Term Memory (LSTM), or the like. Other artificial neural networks may be used in some examples. A classifier may be used instead of a neural network in some examples A classifier may not include hidden layers, but may classify a particular input as corresponding to a particular output. For example, for a set of pressure and/or flow data, an identification of CV or not CV may be generated by the classifier.

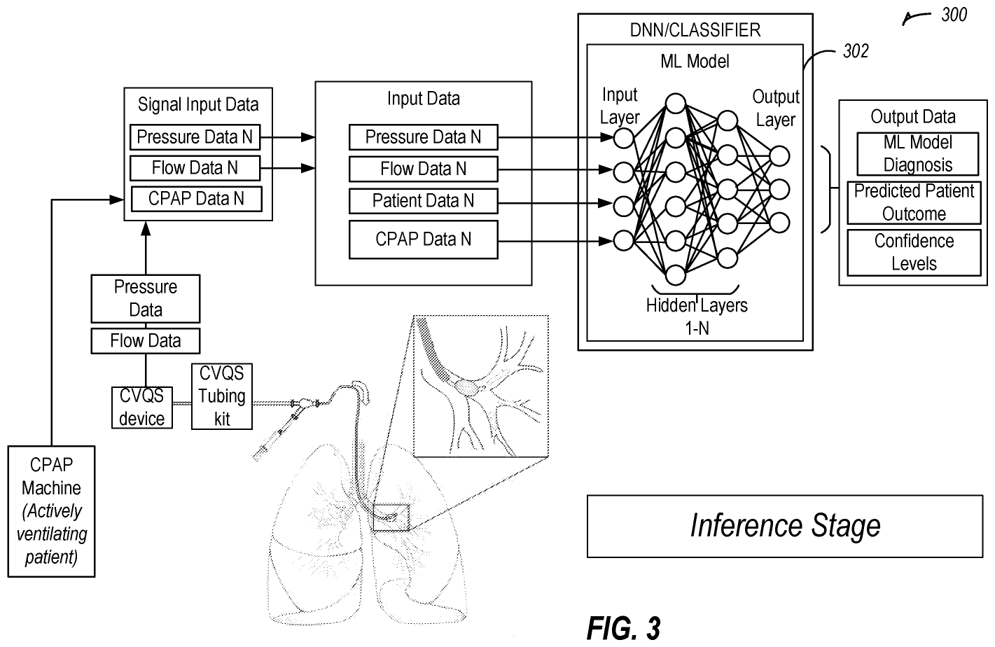

The input data for training the model 202 may include data captured from a CVQS (e.g., CVQS 100 of FIG. 1), with labeled data from a medical practitioner and/or patient. The model 202 may be used in an inference stage (described in further detail below with respect to FIG. 3) for determining a presence (or lack thereof) of collateral ventilation and/or to indicate whether a patient is a candidate for an endoluminal valve.

As shown in FIG. 2, training data may include signal training data that is comprised of measured signals representing quantifiable measurements taken by a flow meter and/or pressure sensor of the CVQS. For example, the training data may include measurements of air flow into and/or out of a lung lobe that is being occluded by the CVQS and that is being considered for treatment by way of endoluminal valve placement into one or more airways that are in fluid communication with the occluded lung lobe. It will be appreciated that by virtue of the lung lobe being occluded, air flow into and/or out of the occluded lobe via the occluded air passage will pass entirely through a lumen of the CVQS that bypasses the occlusion device (e.g., assuming the occlusion device forms a perfect seal which as a practical matter may not always occur in real life settings). The signal training data may include data provided by a CPAP machine (e,g., the constant pressure air supply 112 of FIG. 1) that is actively ventilating a patient. In some examples, the training data may include annotation training data that is provided by a medical practitioner and/or a patient (e.g., as labeling data). For example, a medical practitioner may annotate a patient-specific CVQS data set as being CV present or CV not present and/or annotate a patient specific CVQS data set as representing an assessed degree of CV and/or collateral resistance (Rcoll). This annotated training data may be used to train the model 202. Additionally, or alternatively, a patient-specific CVQS data set may be annotated with subjective patient outcome feedback after having a valve placed or not. For example, an individual patient may be assessed via operation of the CVQS to generate a patient-specific CVQS data set.

Then, subsequent to this assessment, one or more endoluminal valves may be placed within the patient and following this valve placement procedure (e.g., 1 week post valve placement, 1 month post valve placement, etc.) the patient may provide subjective feedback of a perceived improvement or lack thereof. The patient outcome feedback may include a sliding scale value of perceived life improvement resulting from the valve placement (e.g., on a scale of 1-10, how has your previously reported shortness of breath improved following the valve placement procedure).

Based on the signal training data and/or annotation training data, the model 202 may generate output weights corresponding to individual processing nodes that are spread across an input later, an output layer, and one or more hidden layers. The model 202 and trained weights may later be used to infer an indication of CV+or CV−, an indication of a degree of CV, and/or patient suitability for a treatment of a predetermined type (e.g., endoluminal valve placement) based on new inputs from a patient under consideration.

FIG. 3 illustrates a machine learning model inference diagram 300 in accordance with at least one example of this disclosure. In the inference diagram 300, a model 302 (e.g., the model 202 after training, and/or as updated, etc.) may be used to output a prediction, such as whether CV is present for a patient, whether a treatment is recommended (e.g., an endoluminal valve), or the like. A confidence level and/or weighting may be output as the prediction, or in addition to other predictions discussed above, The machine learning model inference diagram 300 may represent an exemplary computer-based clinical decision support system (CDSS) that is configured to assist in predicting a patient-specific outcome that will result from placement of one or more endoluminal valves into one or more bronchial airways that are in fluid communication with a diseased lung portion of a COPD patient.

As shown in FIG. 3, the model 302 may receive signals from a CVQS (e.g., CVQS 100 of FIG. 1) as input, such as pressure data, flow data, and/or data provided by a CPAP machine (e.g., the constant pressure air supply 112 of FIG. 1), and/or other data such as patient data. The model 302 may generate an output (e.g., an inference) that includes a patient suitability assessment (e.g., CV present or no CV present), a predicted patient outcome (e.g., an indication of a likely patient outcome of performing valve placement based on the currently observed input signals), a confidence level (e.g., 95% confident of a “no CV present” patient suitability assessment, 95% confident that valve placement will result in reduced shortness of breath, etc.), an estimated amount of CV, or the like. The model 302 may have a run-time that occurs while a patient is undergoing the CVQS procedure and/or shortly after completion. The model 302 may provide a physician with a Rapid On-Site Evaluation of whether the current patient has collateral ventilation, and/or whether the current patient may benefit from a valve placement into the occluded lobe.

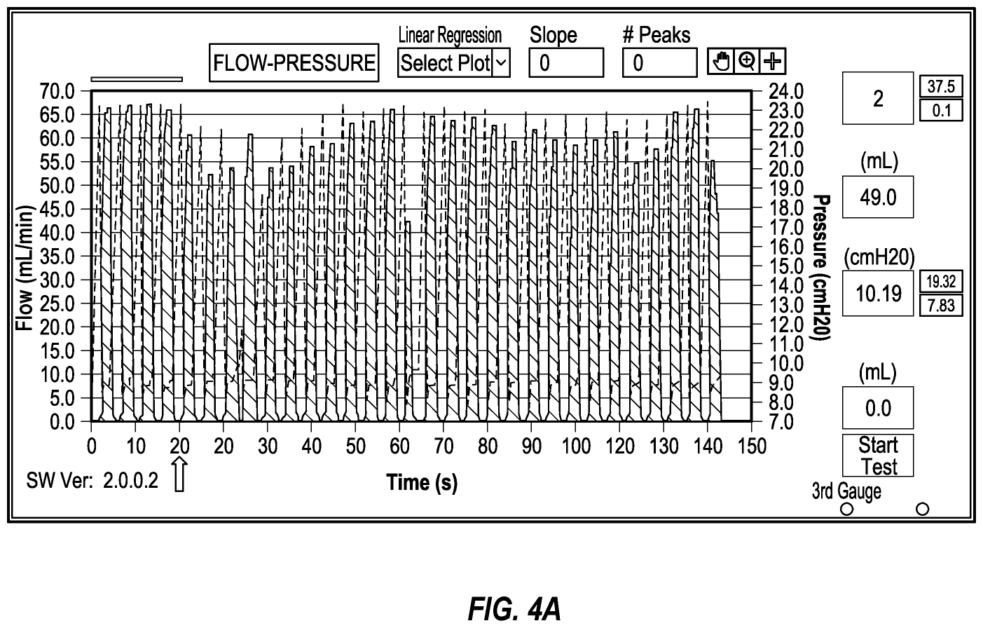

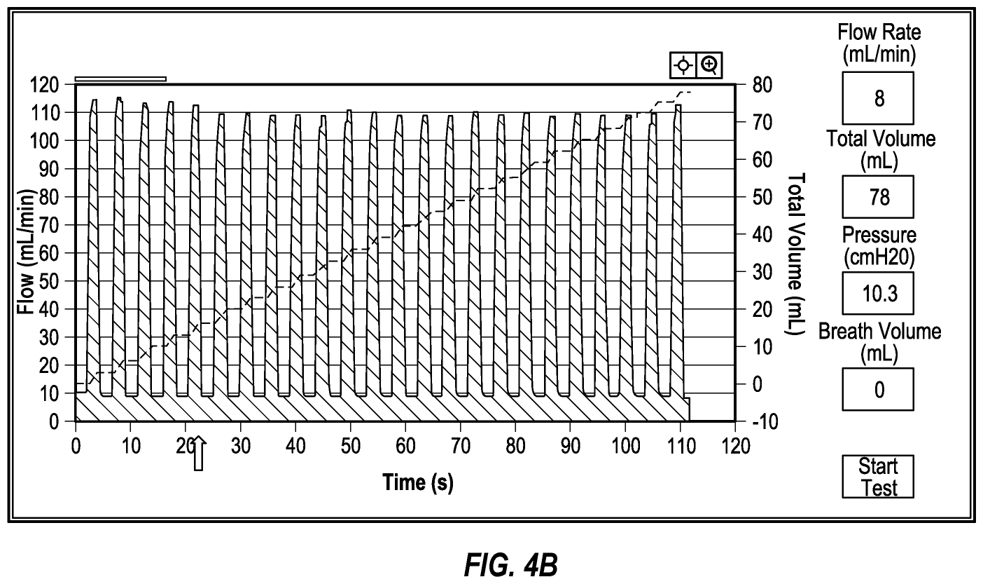

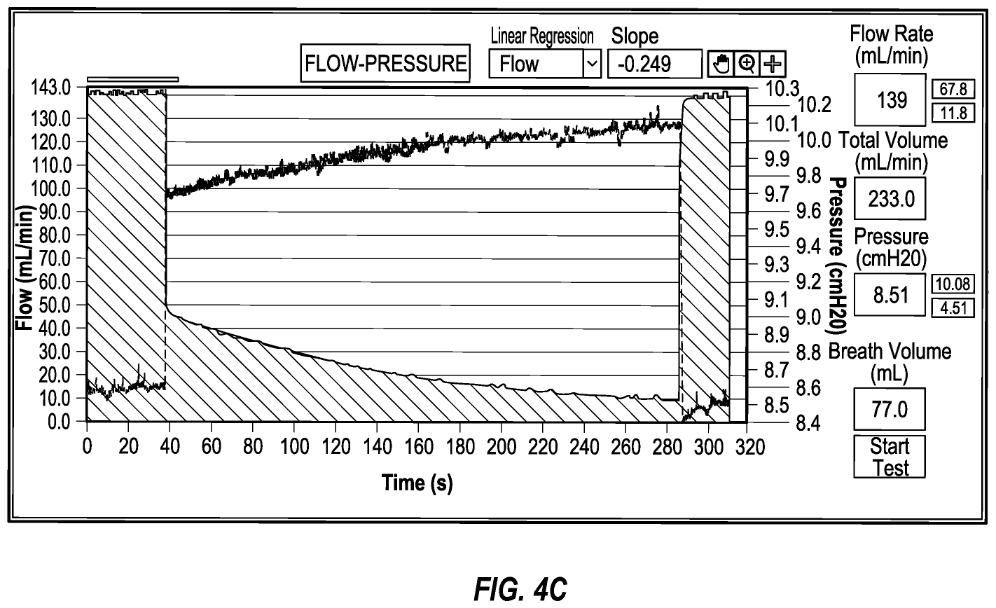

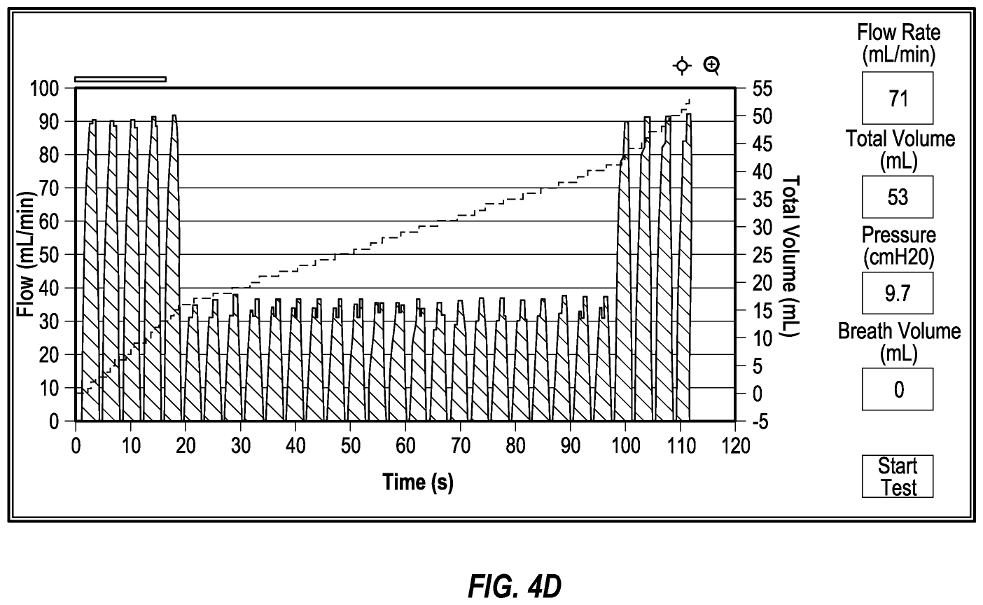

FIG. 4A illustrates a flow and pressure diagram showing an example of collateral ventilation in accordance with at least one example of this disclosure. FIG. 4A includes an arrow indicating whether balloon occlusion occurred (e.g., at 20 seconds). An indication of CV is not readily visible to an untrained eye, and may not be detectable in some examples to a trained physician. In an example, the output graph shown in FIG. 4A may be used as an input to a machine learning trained model to determine whether a corresponding patient has CV. The model may use a classifier and/or neural network to determine if CV is present from the graph and/or from the underlying data.

FIG. 4B illustrates a flow and volume diagram showing an example of collateral ventilation in accordance with at least one example of this disclosure. The vertical graph portions represent flow data and the increasing line represents total volume of flow.

FIG. 4C illustrates a flow and pressure diagram showing an example with a lack of collateral ventilation (no CV present) in accordance with at least one example of this disclosure. FIG. 4D illustrates a flow and volume diagram showing an example with a lack of collateral ventilation (no CV present) in accordance with at least one example of this disclosure. When collateral ventilation is not present, air flow subsides and/or ceases as the pressure within the occluded lobe reaches the source pressure. In the event the ventilator is placed on pause while performing the assessment, air flows into the lobe continuously until pressurized to the CPAP pressure. The graphs shown in FIGS. 4C and 4D represent (CV negative, no CV present) conditions.

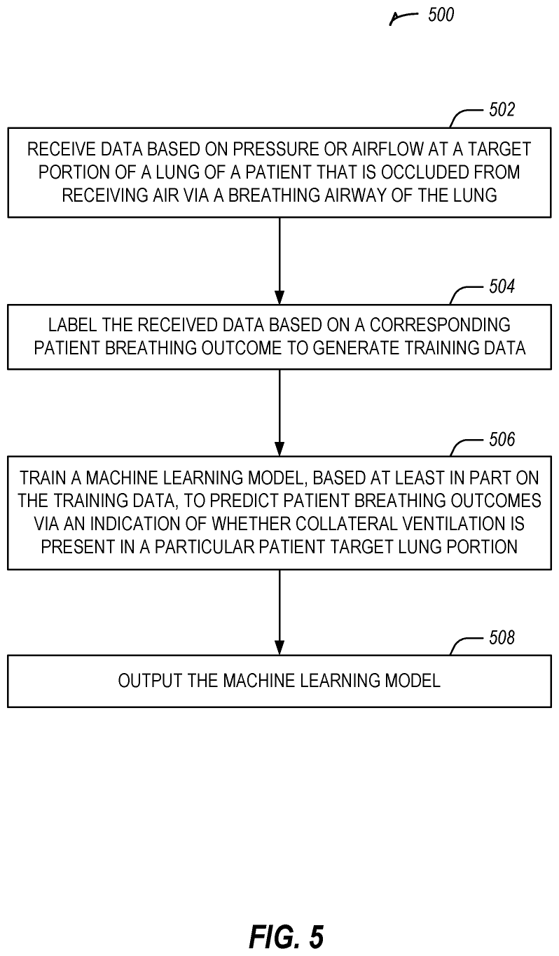

FIG. 5 illustrates a flowchart showing a technique 500 for training a model to determine whether a patient is a candidate for receiving an endoluminal valve based on collateral ventilation data in accordance with at least one example of this disclosure. The technique 500 may be performed by a processor by executing instructions stored in memory.

The technique 500 includes an operation 502 to receive data, for example captured by a sensor, based on pressure and/or airflow at a target portion of a lung of a patient that is occluded from receiving air via a breathing airway of the lung. The occluded breathing airway may be occluded by a balloon to block an outflow airway. In this example, the received data may be pressure data, such as based on an applied positive pressure to an inflow airway. The applied positive pressure may include a constant pressure, for example a constant pressure of 10 cm H2O. In some examples, the occluded breathing airway is occluded by a valve to block an inflow airway while allowing outflow air, and wherein the received data is outflow air data. In an example, operation 502 includes periodically obtaining measurement data of the airflow and/or the pressure at the target portion of the lung.

The technique 500 includes an operation 504 to label the received data based on a corresponding patient breathing outcome to generate training data and/or receiving labeled data. In an example, the corresponding patient breathing outcome includes a clinician determination of whether the patient has collateral ventilation at the target portion of the lung based on the received data. In another example, the corresponding patient breathing outcome includes an objective metric of breathing of the patient and/or a patient reported breathing assessment obtained after a procedure to insert an endoluminal valve in the patient. In some examples, a combination of corresponding patient breathing outcomes may be used.

The technique 500 includes an operation 506 to train a machine learning model, based at least in part on the training data, to predict patient breathing outcomes via an indication of whether collateral ventilation is present in a particular patient target lung portion. Operation 506 may include using at least one of volume data, a medical image of the patient, a fissure integrity score, a disease state of the patient, a patient age, and/or a comorbidity of the patient as additional input data. The indication may include a binary display of either collateral ventilation positive or collateral ventilation negative (e.g., via a light, such as a green light for CV positive or a red light for CV negative, via a user interface displaying text and/or an image, etc.). The indication may include a likelihood of the patient being collateral ventilation positive, a confidence level, or the like.

The technique 500 includes an operation 508 to output the machine learning model. Operation 508 may include deploying the machine learning model (e.g., making the machine learning model available via an API, the internet, via download, etc.), saving the machine learning model (e.g., for later retrieval for use and/or updating), sending the machine learning model to a destination (e.g., to a database and/or server), or the like.

The technique 500 may include an operation to occlude the breathing airway of the target portion of the lung (e.g., using a valve, a balloon, etc.).

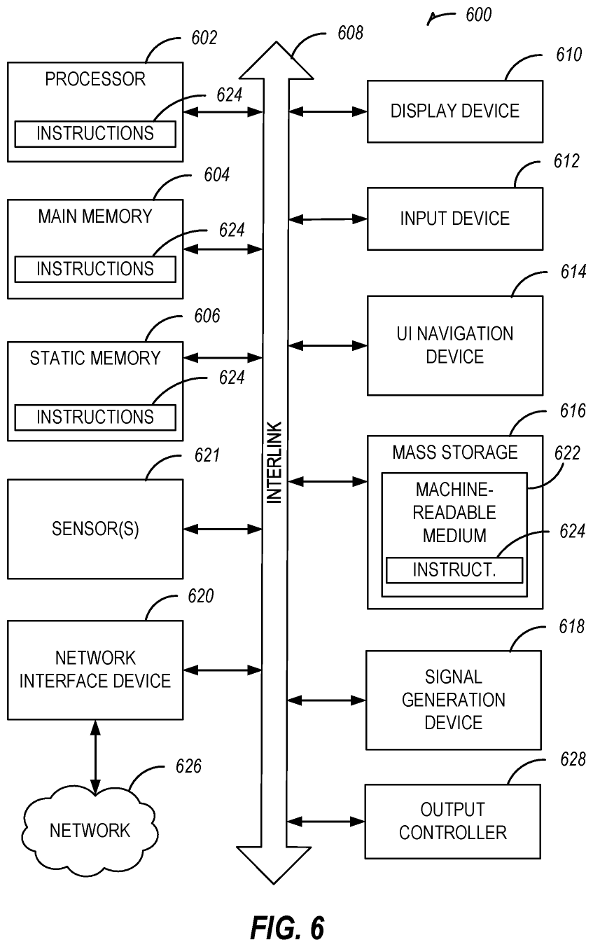

FIG. 6 illustrates a block diagram of an example machine 600 upon which any one or more of the techniques discussed herein may perform in accordance with some embodiments In alternative embodiments, the machine 600 may operate as a standalone device and/or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610, an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensor. The machine 600 may include an output controller 628, such as a serial (e.g., Universal Serial Bus (USB), parallel, or other wired or wireless (e.g., infrared (IR), near field communication (NFC), etc.) connection to communicate and/or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine readable media.

While the machine readable medium 622 is illustrated as a single medium, the term “machine readable medium” may include a single medium or multiple media (e.g., a centralized or distributed database, and/or associated caches and servers) configured to store the one or more instructions 624. The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, and optical and magnetic media,

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

Each of the following non-limiting examples may stand on its own, or may be combined in various permutations or combinations with one or more of the other examples.

Example 1 is a collateral ventilation quantification system for training a machine learning model for use in a computer-based clinical decision support system to assist in predicting patient outcome for endoluminal valve placement, the collateral ventilation quantification system comprising: at least one sensor to capture data based on at least one of pressure or airflow at a target portion of a lung of a patient that is occluded by a device from receiving air via a breathing airway of the lung; processing circuitry; and memory, including instructions, which when executed by the processing circuitry, cause the processing circuitry to perform operations comprising: labeling the received data based on a corresponding patient breathing outcome to generate training data; and training a machine learning model, based at least in part on the training data, to predict one or more patient breathing outcomes via an indication of whether collateral ventilation is present in a particular patient target lung portion; and storing the machine learning model.

Example 2 is a method for training a machine learning model for use in a computer-based clinical decision support system to assist in predicting patient outcome for endoluminal valve placement, the method comprising: receiving data, captured by at least one sensor, that indicates at least one of pressure or airflow at a target portion of a lung of a patient that is occluded by a device from receiving air via a breathing airway of the lung; labeling the received data based on a corresponding patient breathing outcome to generate training data; and training a machine learning model, based at least in part on the training data, to predict one or more patient breathing outcomes via an indication of whether collateral ventilation is present in a particular patient target lung portion; and outputting the machine learning model.

In Example 3, the subject matter of Example 2 includes, wherein the occluded breathing airway is occluded by a balloon to block an outflow airway, and wherein the received data is pressure data based on an applied positive pressure to an inflow airway.

In Example 4, the subject matter of Example 3 includes, wherein the applied positive pressure includes a constant applied pressure.

In Example 5, the subject matter of Examples 2-4 includes, wherein training the machine learning model includes using at least one of volume data of a lung portion, a medical image of the patient, a fissure integrity score, a disease state of the patient, a patient age, or a comorbidity of the patient as additional input data.

In Example 6, the subject matter of Examples 2-5 includes, wherein the corresponding patient breathing outcome includes a clinician determination of whether the patient has collateral ventilation at the target portion of the lung based on the received data.

In Example 7, the subject matter of Examples 2-6 includes, wherein the corresponding patient breathing outcome includes an objective outcome of breathing of the patient or a patient reported breathing assessment obtained after a procedure to insert an endoluminal valve in the patient.

In Example 8, the subject matter of Examples 2-7 includes, wherein the indication of whether collateral ventilation is present in a particular patient target lung portion is output from the model as a binary display of either collateral ventilation being present or collateral ventilation not being present.

In Example 9, the subject matter of Examples 2-8 includes, wherein the indication is output from the model including a probability of the patient having collateral ventilation in the target portion.

In Example 10, the subject matter of Examples 2-9 includes, occluding, using the device, the breathing airway of the target portion of the lung.

In Example 11, the subject matter of Examples 2-10 includes, wherein receiving the data includes recurrently or periodically obtaining measurement data of the airflow or the pressure at the target portion of the lung.

In Example 12, the subject matter of Examples 2-11 includes, wherein the occluded breathing airway is occluded by a valve to block an inflow airway while allowing outflow air, and wherein the received data is outflow air data.

Example 13 is a method for training a machine learning model for use in a computer-based clinical decision support system to assist in predicting patient outcome for endoluminal valve placement, the method comprising: receiving pressure data, captured by at least one sensor, t\hat indicates pressure in a target portion of a lung of a patient that is occluded by a device from receiving air via a breathing airway of the lung; labeling the received pressure data based on a corresponding patient breathing outcome to generate training data; and training a machine learning model, based at least in part on the training data, to predict one or more patient breathing outcomes via an indication of whether collateral ventilation is present in a particular patient target lung portion; and storing the machine learning model.

Example 14 is a method for training a machine learning model for use in a computer-based clinical decision support system to assist in predicting patient outcome for endoluminal valve placement, the method comprising: receiving airflow data, captured by at least one sensor, that indicates airflow out of a target portion of a lung of a patient that is occluded by a device from receiving air via a breathing airway of the lung; labeling the received airflow data based on a corresponding patient breathing outcome to generate training data; and training a machine learning model, based at least in part on the training data, to predict one or more patient breathing outcomes via an indication of whether collateral ventilation is present in a particular patient target lung portion; and storing the machine learning model.

Example 15 is a device for training a machine learning model for use in a computer-based clinical decision support system to assist in predicting patient outcome for endoluminal valve placement, the device comprising: processing circuitry; and memory, including instructions, which when executed by the processing circuitry, cause the processing circuitry to perform operations comprising: receiving data, captured by at least one sensor, that indicates pressure or airflow at a target portion of a lung of a patient that is occluded by a device from receiving air via a breathing airway of the lung; labeling the received data based on a corresponding patient breathing outcome to generate training data; and training a machine learning model, based at least in part on the training data, to predict one or more patient breathing outcomes via an indication of whether collateral ventilation is present in a particular patient target lung portion; and outputting the machine learning model.

Example 16 is at least one machine-readable medium including instructions, which when executed by processing circuitry, cause the processing circuitry to perform operations comprising receiving data, captured by at least one sensor, that indicates pressure or airflow at a target portion of a lung of a patient that is occluded by a device from receiving air via a breathing airway of the lung; labeling the received data based on a corresponding patient breathing outcome to generate training data; and training a machine learning model, based at least in part on the training data, to predict one or more patient breathing outcomes via an indication of whether collateral ventilation is present in a particular patient target lung portion; and outputting the machine learning model.

Example 17 is a method comprising: receiving data, captured by at least one sensor, that indicates at least one of pressure or airflow at a target portion of a lung of a patient that is occluded by a device from receiving air via a breathing airway of the lung; implementing a machine learning model, trained at least in part based on training data including input previous patient sensor data and labeled corresponding previous patient breathing outcomes, to predict a patient breathing outcome for the patient; and outputting an indication of at least one of whether collateral ventilation is present or whether the predicted patient breathing outcome corresponds to placement of an endoluminal valve in the patient based on the prediction from the machine learning model.

In Example 18, the subject matter of Example 17 includes, wherein outputting the indication includes identifying that collateral ventilation is present, and in response, displaying a recommendation to treat the patient with the endoluminal valve.

In Example 19, the subject matter of Examples 17-18 includes, wherein outputting the indication includes identifying that collateral ventilation is not present, and in response, displaying a recommendation to not treat the patient with the endoluminal valve.

In Example 20, the subject matter of Examples 17-19 includes, wherein the occluded breathing airway is occluded by a balloon to block an outflow airway, and wherein the received data is pressure data based on an applied positive pressure to an inflow airway.

In Example 21, the subject matter of Example 20 includes, wherein the applied positive pressure includes a constant applied pressure.

In Example 22, the subject matter of Examples 17-21 includes, wherein outputting the indication includes outputting a probability of the patient having collateral ventilation in the target portion.

In Example 23, the subject matter of Examples 17-22 includes, occluding, using the device, the breathing airway of the target portion of the lung.

In Example 24, the subject matter of Examples 17-23 includes, wherein receiving the data includes recurrently or periodically obtaining measurement data of the airflow or the pressure at the target portion of the lung.

In Example 25, the subject matter of Examples 17-24 includes, wherein the occluded breathing airway is occluded by a valve to block an inflow airway while allowing outflow air, and wherein the received data is outflow air data.

Example 26 is at least one machine-readable medium including instructions that, when executed by processing circuitry, cause the processing circuitry to perform operations to implement of any of Examples 1-25.

Example 27 is an apparatus comprising means to implement of any of Examples 1-25.

Example 28 is a system to implement of any of Examples 1-25.

Example 29 is a method to implement of any of Examples 1-25.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

What is claimed is:

1. A collateral ventilation quantification system for training a machine learning model for use in a computer-based clinical decision support system to assist in predicting patient outcome for endoluminal valve placement, the collateral ventilation quantification system comprising:
at least one sensor to capture data based on at least one of pressure or airflow at a target portion of a lung of a patient that is occluded by a device from receiving air via a breathing airway of the lung;
processing circuitry; and
memory, including instructions, which when executed by the processing circuitry, cause the processing circuitry to perform operations comprising:
labeling the received data based on a corresponding patient breathing outcome to generate training data; and
training a machine learning model, based at least in part on the training data, to predict one or more patient breathing outcomes via an indication of whether collateral ventilation is present in a particular patient target lung portion; and
storing the machine learning model.

2. A method for training a machine learning model for use in a computer-based clinical decision support system to assist in predicting patient outcome for endoluminal valve placement, the method comprising:
receiving data, captured by at least one sensor, that indicates at least one of pressure or airflow at a target portion of a lung of a patient that is occluded by a device from receiving air via a breathing airway of the lung;
labeling the received data based on a corresponding patient breathing outcome to generate training data; and
training a machine learning model, based at least in part on the training data, to predict one or more patient breathing outcomes via an indication of whether collateral ventilation is present in a particular patient target lung portion; and
outputting the machine learning model.

3. The method of claim 2, wherein the occluded breathing airway is occluded by a balloon to block an outflow airway, and wherein the received data is pressure data based on an applied positive pressure to an inflow airway.

4. The method of claim 3, wherein the applied positive pressure includes a constant applied pressure.

5. The method of claim 2, wherein training the machine learning model includes using at least one of volume data of a lung portion, a medical image of the patient, a fissure integrity score, a disease state of the patient, a patient age, or a comorbidity of the patient as additional input data.

6. The method of claim 2, wherein the corresponding patient breathing outcome includes a clinician determination of whether the patient has collateral ventilation at the target portion of the lung based on the received data.

7. The method of claim 2, wherein the corresponding patient breathing outcome includes an objective outcome of breathing of the patient or a patient reported breathing assessment obtained after a procedure to insert an endoluminal valve in the patient.

8. The method of claim 2, wherein the indication of whether collateral ventilation is present in a particular patient target lung portion is output from the model as a binary display of either collateral ventilation being present or collateral ventilation not being present.

9. The method of claim 2, wherein the indication is output from the model including a probability of the patient having collateral ventilation in the target portion.

10. The method of claim 2, further comprising occluding, using the device, the breathing airway of the target portion of the lung.

11. The method of claim 2, wherein receiving the data includes recurrently or periodically obtaining measurement data of the airflow or the pressure at the target portion of the lung.

12. The method of claim 2, wherein the occluded breathing airway is occluded by a valve to block an inflow airway while allowing outflow air, and wherein the received data is outflow air data.

13. A method comprising:
receiving data, captured by at least one sensor, that indicates at least one of pressure or airflow at a target portion of a lung of a patient that is occluded by a device from receiving air via a breathing airway of the lung;
implementing a machine learning model, trained at least in part based on training data including input previous patient sensor data and labeled corresponding previous patient breathing outcomes, to predict a patient breathing outcome for the patient; and
outputting an indication of at least one of whether collateral ventilation is present or whether the predicted patient breathing outcome corresponds to placement of an endoluminal valve in the patient based on the prediction from the machine learning model.

14. The method of claim 13, wherein outputting the indication includes identifying that collateral ventilation is present, and in response, displaying a recommendation to treat the patient with the endoluminal valve.

15. The method of claim 13, wherein outputting the indication includes identifying that collateral ventilation is not present, and in response, displaying a recommendation to not treat the patient with the endoluminal valve.

16. The method of claim 13, wherein the occluded breathing airway is occluded by a balloon to block an outflow airway, and wherein the received data is pressure data based on an applied positive pressure to an inflow airway.

17. The method of claim 16, wherein the applied positive pressure includes a constant applied pressure.

18. The method of claim 13, wherein outputting the indication includes outputting a probability of the patient having collateral ventilation in the target portion.

19. The method of claim 13, further comprising occluding, using the device, the breathing airway of the target portion of the lung.

20. The method of claim 13, wherein receiving the data includes recurrently or periodically obtaining measurement data of the airflow or the pressure at the target portion of the lung.
